(19) United States
(12) Patent Application Publication
(10) Pub. No.: US 2017/0325750 A1
(43) Pub. Date: Nov. 16, 2017

(54) HEART RATE ESTIMATING APPARATUS, HEART RATE ESTIMATING SYSTEM AND HEART RATE ESTIMATING METHOD

(71) Applicant: FUJITSU LIMITED, Kawasaki-shi (JP)

(72) Inventor: Satoshi Tanabe, Meguro (JP)

(73) Assignee: FUJITSU LIMITED, Kawasaki-shi (JP)

(21) Appl. No.: 15/488,577

(22) Filed: Apr. 17, 2017

(30) Foreign Application Priority Data
May 16, 2016 (JP) 2016-097601

(51) Int. Cl. A61B 5/00; A61B 5/024

(52) U.S. Cl. CPC A61B 5/7278; A61B 5/7246; A61B 5/02438; A61B 5/681

(57) ABSTRACT

In a heart rate estimating apparatus, a sensor generates pulse wave data indicating a pulse wave of a body. A storage unit stores a plurality of state transition models each representing states including a normal state and an abnormal state corresponding to noise, transition probabilities between the states, and output probabilities of a plurality of different symbols, wherein at least one of the transition probabilities and the output probabilities differs between the respective state transition models. An estimating unit generates a symbol string that indicates a time series of changes in the pulse wave from the pulse wave data, calculates a fit between the symbol string and each of the plurality of state transition models, and calculates a heart rate estimated from the pulse wave data based on a state transition model selected in accordance with the fit.

### CROSS-REFERENCE TO RELATED APPLICATION

This application is based upon and claims the benefit of priority of the prior Japanese Patent Application No. 2016-097601, filed on May 16, 2016, the entire contents of which are incorporated herein by reference.

### FIELD

The present embodiments discussed herein are related to a heart rate estimating apparatus, a heart rate estimating system, and a heart rate estimating method.

### BACKGROUND

In recent years, apparatuses that estimate the heart rate of the body have been used in various fields, such as sports, healthcare, and medicine. Some apparatuses that estimate the heart rate are wearable terminals, such as wristbands. Heart rate can be estimated by detecting a pulse wave using a pulse wave sensor, such as an optical sensor that measures volumetric changes in blood vessels, and analyzing the periodicity of the pulse wave.

As one example, a pulse wave measuring apparatus that calculates a frequency spectrum of pulse wave data, which has been outputted from a pulse wave sensor, by a fast Fourier transform (FFT) and estimates the heart rate based on a peak frequency in the frequency spectrum has been proposed. On determining that the pulse wave data in a specified measurement segment is abnormal due to the effects of noise, the proposed pulse wave measuring apparatus corrects the estimated heart rate using past pulse wave data that was determined to be normal.

As one example, a signal analyzing method that divides an electrocardiogram signal in a time series into a plurality of segments (this process is called “segmentation”) using a hidden Markov model has also been proposed. As another example, a pulsation detecting apparatus that performs a frequency analyzing process on a pulse wave signal outputted from a pulse wave sensor and estimates the heart rate based on the analysis result has also been proposed. When the signal-to-noise ratio (or “S/N”) of a pulse wave signal is low, the proposed pulsation detecting apparatus performs a Fourier transform as the frequency analyzing process, while when the S/N of the pulse wave signal is high, the pulsation detecting apparatus estimates power spectral density as the frequency analyzing process.

See, for example, Japanese Laid-open Patent Publication No. 2003-265422, International Publication Pamphlet No. WO2005/107587, and International Publication Pamphlet No. WO2013/145731.

There are cases where noise is included in the pulse wave data outputted from a pulse wave sensor due to various factors, such as changes in the positional relationship between the body and the pulse wave sensor due to the body moving. When estimating the heart rate from pulse wave data with lots of noise, there is a drop in estimation precision compared to when the heart rate is estimated from pulse wave data with little noise. As a method of reducing the effects of noise, it would be conceivable to extend the measurement period of the pulse wave and estimate the heart rate from pulse wave data for a longer period. However, it is not desirable to extend the measurement time due to reasons such as an increase in power consumption by a pulse wave sensor.

### SUMMARY

According to one aspect, there is provided a heart rate estimating apparatus including: a sensor that generates pulse wave data indicating a pulse wave of a body; a memory that stores a plurality of state transition models, the plurality of state transition models each representing a plurality of states including a normal state and an abnormal state corresponding to noise, transition probabilities between the plurality of states, and output probabilities of each of a plurality of different symbols being observed in each of the plurality of states, wherein at least one of the transition probabilities and the output probabilities differs between the respective state transition models; and a processor that generates a symbol string that indicates a time series of changes in the pulse wave from the pulse wave data using the plurality of different symbols, calculates a fit between the symbol string and each of the plurality of state transition models, and calculates a heart rate estimated from the pulse wave data based on a state transition model that has been selected out of the plurality of state transition models in accordance with the fit.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 depicts one example of a heart rate estimating apparatus according to a first embodiment;

FIG. 2 depicts one example of a system including a heart rate estimating apparatus and information processing apparatuses according to a second embodiment;

FIG. 3 depicts one example of hardware capable of realizing the functions of the heart rate estimating apparatus according to the second embodiment;

FIG. 4 depicts one example of hardware capable of realizing the functions of an information processing apparatus according to the second embodiment;

FIG. 5 is a graph depicting one example of pulse wave data observed when (spike type) body movement noise is present;

FIG. 6 is a graph depicting one example of pulse wave data observed when (continuous, low frequency) body movement noise is present;

FIG. 7 is a block diagram depicting one example of the functions of the heart rate estimating apparatus according to the second embodiment;

FIG. 8 is a flowchart depicting an operation (or “heart rate estimating process”) of the heart rate estimating apparatus according to the second embodiment;

FIG. 9 is a flowchart depicting an operation (or “label series generation process”) of the heart rate estimating apparatus according to the second embodiment;

FIG. 10 is a graph depicting one example of a model waveform used to calculate a correlation value;

FIG. 11 depicts the relationship between the pulse wave data and the correlation value;

FIG. 12 depicts one example of a classification method based on a frequency distribution relating to the correlation value;

FIG. 13 depicts how the correlation value is affected by the presence or absence of baseline fluctuation noise;

FIG. 14 is a state transition chart that defines a hidden Markov model used to estimate heart rate;

FIG. 15 depicts the relationship between the pulse wave data, the correlation value, a label series, and states (when there is little body movement noise);

FIG. 16 depicts the relationship between the pulse wave data, the correlation value, the label series, and the states (when there is upwardly spiking body movement noise);

FIG. 17 depicts the relationship between the pulse wave data, the correlation value, the label series, and the states (when there is downwardly spiking body movement noise);

FIG. 18 is a flowchart depicting the operation (or “generation process for model information”) of the heart rate estimating apparatus according to the second embodiment; and

FIG. 19 depicts another example of a system according to the second embodiment.

### DESCRIPTION OF EMBODIMENTS

Several embodiments will be described below with reference to the accompanying drawings, wherein like reference numerals refer to like elements throughout.

### 1. First Embodiment

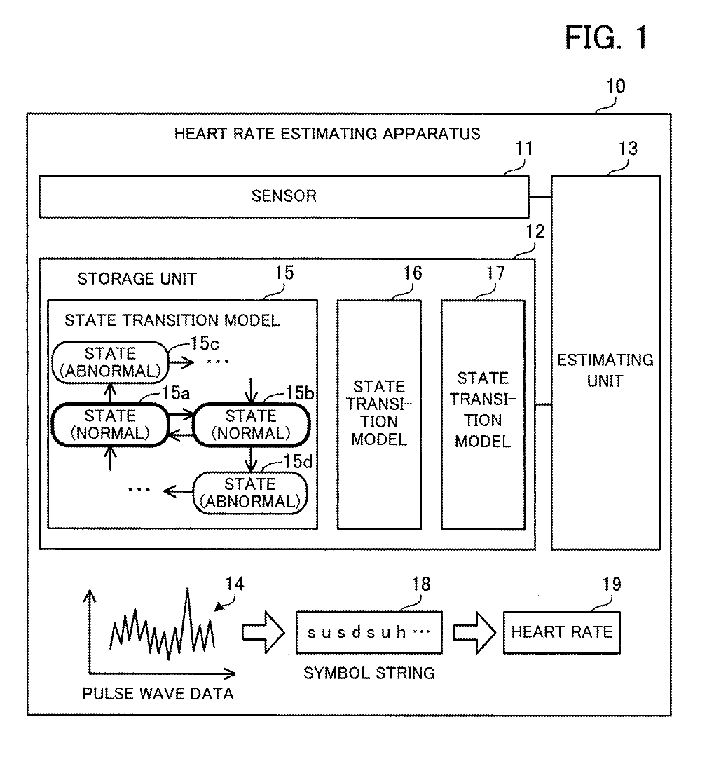

A first embodiment will now be described. FIG. 1 depicts one example of a heart rate estimating apparatus 10 according to a first embodiment.

The heart rate estimating apparatus 10 according to the first embodiment estimates the heart rate of a body.

As one example, the heart rate estimating apparatus 10 is a wearable device, such as a wristband-type device that is attached to the human wrist. The heart rate estimating apparatus 10 includes a sensor 11, a storage unit 12, and an estimating unit 13.

Here, although the heart rate estimating apparatus 10 in this first embodiment includes the sensor 11, the sensor 11 may be provided in a device that is external to the heart rate estimating apparatus 10. For example, a wearable device provided externally to the heart rate estimating apparatus 10 may include the sensor and the heart rate estimating apparatus 10 may communicate wirelessly or via wires with the external wearable device. In this configuration, the heart rate estimating apparatus 10 may be any information processing apparatus such as a mobile phone, a smartphone, a tablet computer, a notebook computer, or a server computer.

The sensor 11 generates pulse wave data 14 indicating the pulse wave of a body. As one example, the sensor 11 is an optical pulse wave sensor that measures volumetric changes in blood vessels in the wrist based on changes in the absorption of light. However, it is also possible to use a different type of sensor, such as an electrical signal sensor.

The pulse wave data 14 may include noise that causes the pulse wave to change due to causes aside from the heart rate. As one example, the pulse wave data 14 may include noise whose value suddenly rises or falls momentarily. As another example, the pulse wave data 14 may include noise (or “baseline fluctuation noise”) where the moving average gradually rises or falls over several seconds or longer. As one example, noise may be produced when the distance between the body and the sensor 11 changes due to the body moving.

The storage unit 12 stores a plurality of state transition models, including state transition models 15 to 17. The storage unit 12 may be a volatile semiconductor memory, such as RAM (Random Access Memory), or may be a nonvolatile storage apparatus, such as flash memory.

The state transition models 15 to 17 each include a plurality of states that include normal states as well as abnormal states that correspond to noise, transition probabilities that are the probabilities of transitions occurring between the plurality of states, and output probabilities that are the probabilities that each out of a plurality of different symbols will be observed in the respective states. The state transition models 15 to 17 may be so-called “hidden Markov models”.

As one example, the state transition models 15 to 17 include states 15a and 15b corresponding to normal states and states 15c and 15d corresponding to abnormal states. When the pulse wave data 14 does not include noise, it is assumed that transitions that cycle between the state 15a and the state 15b that are normal states will occur. As one example, the state 15a corresponds to a period where the value is rising and the state 15b corresponds to a period where the value is falling. One cycle of the pulse wave corresponds to one round of transitions between the states 15a and 15b . On the other hand, when the pulse wave data 14 includes noise, it is assumed that there will be a transition from one of the normal states to one of the abnormal states and then a return from the abnormal state to a normal state. As examples, in the states 15c and 15d that are abnormal states, out of the plurality of different symbols, symbols indicating abnormal changes in the pulse wave are more likely to be observed than in the states 15a and 15b that are the normal states.

At least one out of the transition probabilities and the output probabilities differs between the state transition models 15 to 17. As one example, the state transition models 15 to 17 are generated from a plurality of sample pulse wave data with different noise patterns. The state transition models 15 to 17 may also be generated from a plurality of sample pulse wave data with different combinations of noise patterns and heart rates. Each state transition model may be associated with a heart rate (or “model heart rate”) of the sample pulse wave data that was used.

The estimating unit 13 acquires the pulse wave data 14 from the sensor 11 and estimates a heart rate 19 from the acquired pulse wave data 14. The estimating unit 13 may be a processor such as a CPU (Central Processing Unit) or a DSP (Digital Signal Processor). The estimating unit 13 may include application-specific electronic circuitry, such as an ASIC (Application Specific Integrated Circuit) or an FPGA (Field Programmable Gate Array). The processor executes programs stored in a memory such as RAM (which may be the storage unit 12). The programs executed by the processor include a heart rate estimating program in which the processing described below is written. In this specification, a group including a plurality of processors is referred to as a “multi-processor” or simply as a “processor”.

The estimating unit 13 generates a symbol string 18, which indicates a time series of changes in the pulse wave, from the pulse wave data 14 in accordance with a predetermined feature extracting method. A plurality of different symbols whose output probabilities are defined in the state transition models 15 to 17 are used in the symbol string 18. As one example, the estimating unit 13 extracts an index value indicating the amount of change per unit time using a differential filter or the like, and maps the index value of each time onto a symbol. As one example, the plurality of different symbols include a symbol indicating a small amount of change, a symbol indicating a normal increase, a symbol indicating an abnormally large increase, a symbol indicating a normal decrease, and a symbol indicating an abnormally large decrease. Here, the estimating unit 13 may calculate the distribution of appearance frequencies of index values in the pulse wave data 14 and then decide how the index values and symbols are to be associated based on the distribution of the appearance frequencies. That is, it is possible to determine whether an amount of change is normal or abnormal from the relative relationships between the values present in the pulse wave data 14.

The estimating unit 13 calculates the fit between the symbol string 18 and each of the state transition models 15 to 17. The “fit” referred to here is the probability (or “likelihood”) of the symbol string 18 being observed in accordance with transition probabilities and output probabilities. The estimating unit 13 selects one of the state transition models in accordance with the fit. As one example, the estimating unit 13 selects the state transition model with the best fit.

The estimating unit 13 then calculates the heart rate 19 corresponding to the pulse wave data 14 based on the selected state transition model. As one example, the estimating unit 13 sets the model heart rate associated with the selected state transition model as the heart rate 19. Also, as another example, the estimating unit 13 compares the selected state transition model and the symbol string 18, calculates the average time taken for one round of the states 15a and 15b that are normal states, and calculates the heart rate 19 based on the average time.

The heart rate estimating apparatus 10 may store the heart rate 19 in a storage apparatus provided in the heart rate estimating apparatus 10. Also, the heart rate estimating apparatus 10 may output the heart rate 19 from an output apparatus provided in the heart rate estimating apparatus 10. As examples, the heart rate estimating apparatus 10 may display the heart rate 19 on a display provided in the heart rate estimating apparatus 10 and/or may reproduce a voice that reads out the heart rate 19 from a speaker provided in the heart rate estimating apparatus 10. Also, the heart rate estimating apparatus 10 may transmit the heart rate 19 wirelessly or via wires to an external information processing apparatus. The external information processing apparatus may store the heart rate 19 in a storage apparatus and/or output the heart rate 19 from an output apparatus.

With the heart rate estimating apparatus 10 according to the first embodiment, the symbol string 18 is generated from the pulse wave data 14 and the fit is calculated between the symbol string 18 and each of a plurality of state transition models, in which at least one of the transition probabilities and output probabilities differs. The heart rate 19 corresponding to the pulse wave data 14 is then estimated based on the state transition model that is selected in accordance with the calculated fit.

With the above configuration, it is possible to estimate a heart rate 19 with consideration to noise which may occur in a variety of noise patterns. This means that the estimation precision of the heart rate 19 is improved compared to when the heart rate 19 is estimated from a single state transition model that does not include states corresponding to noise. Also, since it is possible to estimate the heart rate 19 directly from a pulse wave that includes noise, it is possible to reduce the measurement period for a pulse wave compared to when the heart rate 19 is estimated by avoiding pulse wave segments with large amounts of noise. As a result, it is possible to reduce the power consumption of the heart rate estimating apparatus 10.

### 2. Second Embodiment

A second embodiment will now be described.

2-1. System

The second embodiment relates to a heart rate estimating apparatus in the form of a band that monitors the heart rate of a body. This heart rate estimating apparatus is one example of a wearable device that is used by being worn on the body. This wearable device is equipped with a wireless communication function and is capable of remotely transmitting information, such as the heart rate, to an information processing apparatus such as a smartphone or a personal computer (PC).

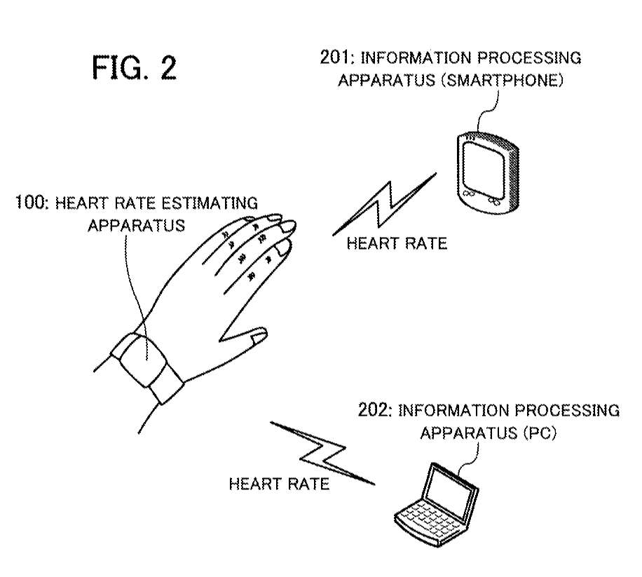

FIG. 2 depicts one example of a system including a heart rate estimating apparatus and information processing apparatuses according to the second embodiment. The system depicted in FIG. 2 includes a heart rate estimating apparatus 100 and information processing apparatuses 201 and 202. The heart rate estimating apparatus 100 is equipped with a sensor that detects a pulse wave and has a function that estimates the heart rate by analyzing data on a pulse wave (or “pulse wave data”).

Note that the expression “pulse wave” refers to volumetric changes in blood vessels caused by the heart pumping blood. The expression “heart rate” refers to the number of volumetric changes in blood vessels in one minute. The pulse wave data is time series data with a periodic waveform. The heart rate estimating apparatus 100 transmits the estimated heart rate by wireless communication to the information processing apparatus 201 (for example, a smartphone) and the information processing apparatus 202 (for example, a PC).

2-2. Hardware

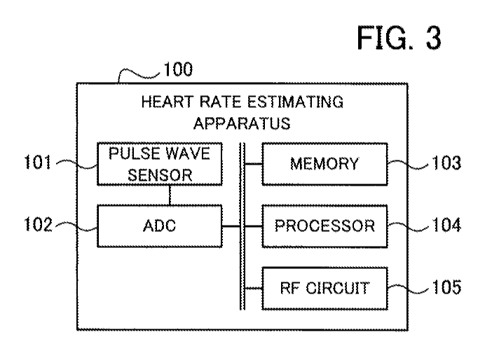

As one example, the functions of the heart rate estimating apparatus 100 may be realized by hardware such as that depicted in FIG. 3. FIG. 3 depicts one example of hardware capable of realizing the functions of the heart rate estimating apparatus according to the second embodiment.

As depicted in FIG. 3, the heart rate estimating apparatus 100 includes a pulse wave sensor 101, an ADC (Analog to Digital Converter) 102, a memory 103, a processor 104, and an RF (Radio Frequency) circuit 105.

The pulse wave sensor 101 is a sensor that detects a pulse wave from a body.

As one example, the pulse wave sensor 101 is an optical pulse wave sensor and detects the pulse wave using absorption of light that changes in accordance with volumetric changes in blood vessels. The ADC 102 is a converter circuit that converts analog data of the pulse wave detected by the pulse wave sensor 101 to digital data.

The memory 103 is a storage element such as SRAM (Static Random Access Memory) or DRAM (Dynamic Random Access Memory). Note that the memory 103 may temporarily store programs executed by the processor 104 and data used in computation. Also, the memory 103 may have a plurality of storage elements and may be a nonvolatile memory.

The processor 104 is a computation circuit such as a CPU, an FPGA (Field-Programmable Gate Array) or an LSI (Large-Scale Integration) element. The processor 104 may include circuits such as adders, multipliers, barrel shifters, and floating point units. The RF circuit 105 is a wireless communication circuit that is connected to an antenna (not illustrated) and controls transmission and reception of RF signals.

Note that the processor 104 is one example of the estimating unit 13 in the first embodiment. The pulse wave sensor 101 is one example of the sensor 11 in the first embodiment. The memory 103 is one example of the storage unit 12 in the first embodiment.

Information Processing Apparatuses

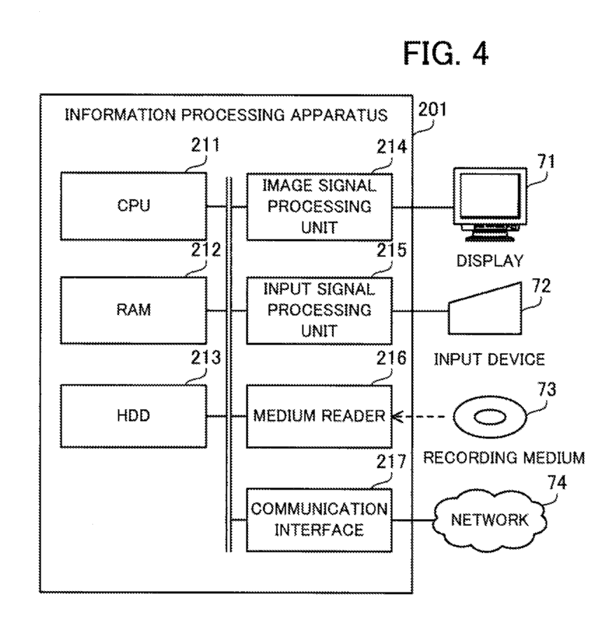

As one example, the functions of the information processing apparatus 201 may be realized by hardware such as that depicted in FIG. 4. FIG. 4 depicts one example of hardware capable of realizing the functions of an information processing apparatus according to the second embodiment.

The information processing apparatus 201 includes a CPU 211, a RAM 212, an HDD (Hard Disk Drive) 213, an image signal processing unit 214, an input signal processing unit 215, a medium reader 216, and a communication interface 217.

The CPU 211 is a processor including computational circuitry that executes instructions of a program. The CPU 211 loads at least part of a program and data stored in the HDD 213 into the RAM 212 and executes the program.

Note that the CPU 211 may include a plurality of processor cores, the information processing apparatus 201 may also include a plurality of processors, and the processing described below may be executed in parallel using a plurality of processors or processor cores. In the following description, a group including a plurality of processors (or so-called “multi-processor”) may be referred to simply as a “processor”.

The RAM 212 is a volatile memory that temporarily stores programs executed by the CPU 211 and data used in computation by the CPU 211. Note that the information processing apparatus 201 may be equipped with other types of memory aside from RAM and may be equipped with a plurality of memories.

The HDD 213 is a nonvolatile storage apparatus that stores software programs, such as an OS (Operating System) and application software, and data. Note that the information processing apparatus 201 may include another type of storage apparatus, such as flash memory or an SSD (Solid State Drive), and may include a plurality of nonvolatile storage apparatuses.

The image signal processing unit 214 outputs images to a display 71 connected to the information processing apparatus 201 in accordance with instructions from the CPU 211. As the display 71, it is possible to use a cathode ray tube (CRT) display, a liquid crystal display (LCD), a plasma display panel (PDP), an organic electro-luminescence (OEL) display, or the like.

The input signal processing unit 215 acquires an input signal from an input device 72 connected to the information processing apparatus 201 and outputs to the CPU 211.

As the input device 72, it is possible to use a pointing device such as a mouse, a touch panel, or a trackball, a keyboard, a remote controller, button switches, or the like. The information processing apparatus 201 may be connected to a plurality of types of input devices. Note that at least one of the display 71 and the input device 72 may be integrally formed with a case of the information processing apparatus 201.

The medium reader 216 is a reader apparatus that reads programs and/or data recorded on a recording medium 73. As examples of the recording medium 73, it is possible to use a magnetic disk such as a flexible disk (FD) or an HDD, an optical disc such as a Compact Disc (CD) and a Digital Versatile Disc (DVD), a magneto-optical disc (MO), or a semiconductor memory. As one example, the medium reader 216 stores programs and data read from the recording medium 73 into the RAM 212 or the HDD 213.

The communication interface 217 is an interface that is connected to a network 74 and communicates with other information processing apparatuses via the network 74. The communication interface 217 may be a wired communication interface connected by a cable to a communication apparatus such as a switch, or may be a wireless communication interface connected by a wireless link to a base station. Note that the functions of the information processing apparatus 202 may also be realized by the hardware depicted in FIG. 4.

2-3 Functions and Operation of Heart Rate Estimating Apparatus

The functions and operation of the heart rate estimating apparatus 100 will now be described.

Regarding Power Consumption

One method of calculating (estimating) the heart rate from pulse wave data is a method that uses a discrete Fourier transform (DFT). In this method, a spectrum is generated using a DFT from pulse wave data and the heart rate is calculated from the peak frequency in the spectrum. The relationship between the heart rate HR and the peak frequency fₘₐₓ is given by Expression (1) below. The relationship between the frequency resolution Δf of the spectrum and the sensor operating time T is given by Expression (2) below. Also, the resolution R of the heart rate is given by Expression (3) below.

$$\begin{matrix}HR = 60 \cdot {f}_{max} & ( 1 ) \\ Δ f = \frac{1}{T} & ( 2 ) \\ R = \frac{1}{Δ HR} = \frac{1}{60 \cdot Δ f} = \frac{T}{60} & ( 3 )\end{matrix}$$

From Expression (3), it can be understood that the longer the sensor operating time T, the higher the resolution R of the heart rate. However, as the sensor operating time T becomes longer, the average power consumed by the sensor increases. Since wearable devices are driven by batteries, it is important for a device to operate for a long time without recharging. As one example, when the resolution R of the heart rate is assumed to 1 bpm (beats per minute), the sensor operating time T is 60 seconds. When the power consumption of an optical pulse wave sensor is assumed to be 5 mW, the energy consumed by the sensor is 300 mJ. As a method of suppressing power consumption, there is a method (Xiangmin Fan, “BayesHeart: Probabilistic Approach for Robust, Low Latency Heart Rate Monitoring on Camera Phones”, In Proc. IUI 2015) of calculating a heart rate from pulse wave data using a hidden Markov model in place of a DFT. With this method, since the resolution of the heart rate is decided by the sampling frequency, a sufficient resolution R for the heart rate is obtained, even when the sensor operating time T is short. As one example, with a sampling frequency of 60 Hz, a resolution R of 1 bpm is obtained. The heart rate estimating apparatus 100 calculates the heart rate from the pulse wave data using a hidden Markov model.

Effect of Body Movement Noise

When estimating the heart rate from the pulse wave data, the heart rate estimating apparatus 100 considers the effect of body movement noise that may be added to pulse wave data due to movement of the body.

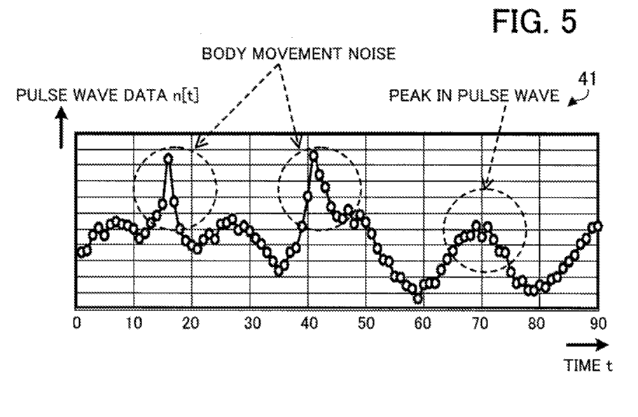

As one example, as depicted in FIG. 5, when the body of the wearer of the heart rate estimating apparatus 100 moves, noise (or “body movement noise”) with a large amplitude may become incorporated in the pulse wave data. FIG. 5 is a graph depicting one example of pulse wave data observed when (spike-type) body movement noise is present. Body movement noise appears in two places in the graph 41 depicted in FIG. 5.

One method of dealing with body movement noise is a method that discards sections (as one example, parts with a width of two seconds or longer) in the periphery of parts of the pulse wave data where body movement noise is included. However, when some of the pulse wave data is discarded, the sensor ends up operating for a longer time to replenish the discarded parts of the pulse wave data, which means that the sensor operating time T becomes longer. As a result, there is an increase in power consumption.

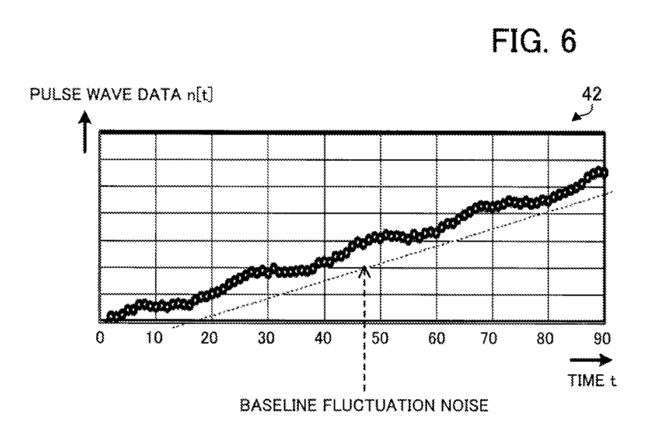

Also, as depicted in FIG. 6, when body movement noise including a large amount of low frequency components (or “baseline fluctuation noise”) is incorporated in the pulse wave data, there is a drop in the estimation precision of the heart rate. FIG. 6 is a graph depicting one example of pulse wave data observed when (continuous, low frequency) body movement noise is present. Rightward-rising baseline fluctuation noise appears in the graph 42 depicted in FIG. 6. The heart rate estimating apparatus 100 according to the second embodiment inputs elements from which the effects of body movement noise have been removed into a hidden Markov model and thereby estimates the heart rate with favorable precision while suppressing an increase in power consumption. The functions and operation of the heart rate estimating apparatus 100 are described below.

Note that in the following description, the pulse wave data measured at time t is expressed as n[t] where t=1, 2, . . . The label series (described later) generated from the pulse wave data n[t] is expressed as x[t], and the hidden Markov model (pulse wave model) used to estimate the heart rate is expressed as Ω[k] where k=1, 2, . . . It is assumed here that the pulse wave model Ω is provided in advance and that information that defines the pulse wave model Ω (model information) is stored by the heart rate estimating apparatus 100.

Function Blocks

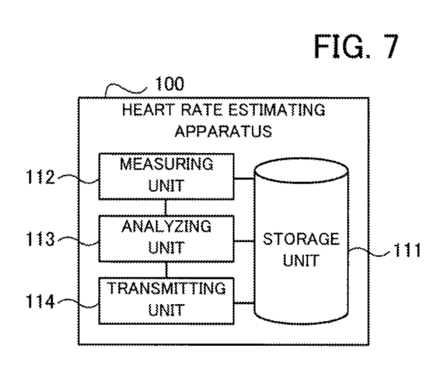

The functions of the heart rate estimating apparatus 100 include elements such as those depicted in FIG. 7. FIG. 7 is a block diagram depicting one example of the functions of the heart rate estimating apparatus according to the second embodiment.

As depicted in FIG. 7, the heart rate estimating apparatus 100 includes a storage unit 111, a measuring unit 112, an analyzing unit 113, and a transmitting unit 114. Note that the functions of the storage unit 111 may be realized by the memory 103 described earlier. The functions of the measuring unit 112 may be realized by the functions of the pulse wave sensor 101 described earlier. The functions of the analyzing unit 113 may be realized by the processor 104 described earlier. The functions of the transmitting unit 114 may be realized by the RF circuit 105 described earlier.

The storage unit 111 stores model information. The measuring unit 112 acquires digital pulse wave data by detecting a pulse wave and performing analog to digital conversion of pulse wave data. The analyzing unit 113 analyzes the pulse wave data acquired by the measuring unit 112 and calculates an estimated value of the heart rate. Note that for ease of explanation, an estimated value of the heart rate may also be referred to below simply as the “heart rate”. Also, calculation of the estimated value of the heart rate may be referred to simply as “estimating the heart rate”. The transmitting unit 114 transmits the heart rate calculated by the analyzing unit 113 to the information processing apparatus 201 or the like.

Heart Rate Estimating Process

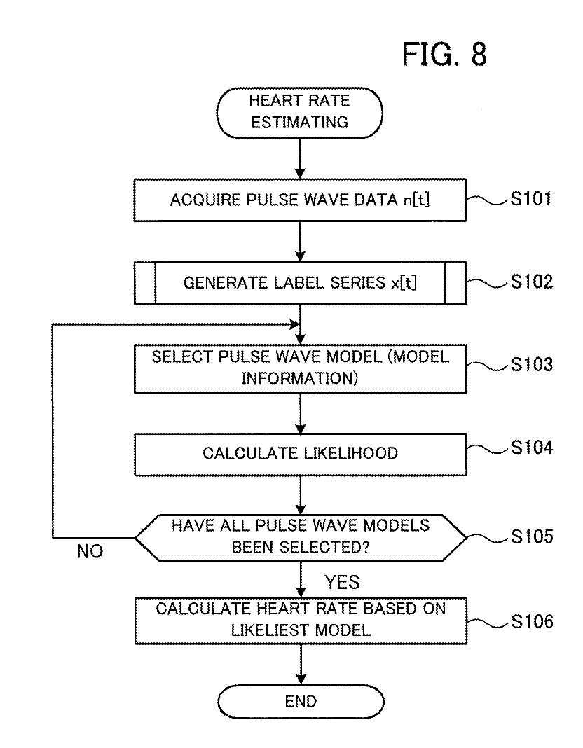

The process that estimates the heart rate is executed by the heart rate estimating apparatus 100 according to the flow depicted in FIG. 8. FIG. 8 is a flowchart depicting an operation (or “heart rate estimating process”) of the heart rate estimating apparatus according to the second embodiment.

(S101) When the process is commenced, the measuring unit 112 acquires the pulse wave data n[t]. That is, detection of the pulse wave by the pulse wave sensor 101 and analog to digital conversion of pulse wave data by the ADC 102 are performed and the pulse wave data n[t] is inputted into the analyzing unit 113.

(S102) The analyzing unit 113 generates a label series x[t] from the pulse wave data n[t]. The label series x[t] includes labels that express features of a waveform of the pulse wave data at the time t. The label series x[t] is time series data expressing the features of the waveform, such as the waveform steeply projecting upward (an upwardly spiking waveform), the waveform steeply projecting downward (a downwardly spiking waveform), and the waveform of typical pulse wave data when there is no body movement noise. The method of generating the label series x[t] is described in more detail later in this specification.

(S103) The analyzing unit 113 selects one pulse wave model Ω that has been prepared in advance. Model information (i.e., model parameters of a hidden Markov model) that defines each pulse wave model Ω is stored in advance in the storage unit 111. The analyzing unit 113 reads the model information of the selected pulse wave model Ω from the storage unit 111.

(S104) The analyzing unit 113 uses the label series x[t] generated in S102 and the model information read out from the storage unit 111 in step S103 to calculate the likelihood of the selected pulse wave model Ω. As one example, when the model information of the pulse wave model Ω is expressed as (A,Q,r) and a state series is expressed as s(t), the likelihood of the pulse wave model Ω is expressed by Expression (4) below. This likelihood expresses the probability of observing a given label series and can be calculated by dynamic programming (for example, a Viterbi algorithm).

$$\begin{matrix}P ( \{ x [ t ] \} ; A , Q , r ) = {\sum }_{s} P ( \{ x [ t ] \} , \{ s [ t ] \} ; A , Q , r ) \\ \approx {max}_{s} [ P ( \{ x [ t ] \} , \{ s [ t ] \} ; A , Q , r ) ]\end{matrix} \qquad ( 4 )$$

Note that when the pulse wave model Ω is expressed by M states S₁, S₂, . . . , s[t]∈{S₁,S₂, . . . }. Also, the model information A of the pulse wave model Ω is expressed by a matrix with transition probabilities P (Sᵢ|Sᵢ) (where i,j=1,2, . . . ) between states as elements. The model information Q is expressed by a matrix with probabilities P(v₁|Sⱼ) of observing the labels v₁ (where l=1,2, . . . ) in the state Sⱼ. The model information r is expressed by a vector that has the probabilities P (Sⱼ) of the initial state (i.e., the state at t=1) being Sⱼ as elements.

As one example, the analyzing unit 113 calculates the likelihood P({x[t]};A,Q,r) using the following method. Note that the number of states is expressed as “N_{c}” and the number of elements in a label series x[t] is expressed as n (that is, i,j=1,2, . . . , N_{c} and t=1,2, . . . , n). First, the analyzing unit 113 set initial values ψ₁(i) and φ₁(i) in accordance with Expression (5) and Expression (6) below. Here, rᵢ is the i^{th} element of vector r. Also, P(x[1]|Sᵢ) is the element in the 1^{st} row and i^{th} column of the matrix Q.

ψ₁(i)=rᵢ·P(x[1]|Sᵢ) (5)

φ₁(i)=0 (6)

Next, the analyzing unit 113 successively calculates ψₜ (j) and φₜ (j) for the t=1,2, . . . , n in accordance with Expression (7) and Expression (8) below. Here, aᵢⱼ is the i^{th} row, j^{th} column element in matrix A. Also, P(x[t]|Sⱼ) is the t^{th} row, j^{th} column element in matrix Q. The analyzing unit 113 then calculates the likelihood P({x[t]};A,Q,r) in accordance with Expression (9) below.

$$\begin{matrix}{Ψ}_{t} ( j ) = {max}_{i} \{ {Ψ}_{t - 1} ( i ) \cdot {a}_{ij} \} \cdot P ( x [ t ] \vert {S}_{j} ) & ( 7 ) \\ {φ}_{t} ( j ) = {arg max}_{i} \{ {Ψ}_{t - 1} ( i ) \cdot {a}_{ij} \} & ( 8 ) \\ P ( \{ x [ t ] \} ; A , Q , r ) = {max}_{i} \{ {Ψ}_{n} ( i ) \} & ( 9 )\end{matrix}$$

(where i,j=1,2, . . . , N_{c}, N_{c} is the number of states, t=2, 3, . . . , n, and n is the number of elements in the label series)

(S105) The analyzing unit 113 determines whether every pulse wave model Ω has been selected. When every pulse wave model Ω has been selected, the pulse wave model Ω with the highest likelihood (hereinafter, “likeliest model”) is specified by the analyzing unit 113 and the processing proceeds to S106. On the other hand, when there is an unselected pulse wave model Ω, the processing proceeds to S103.

(S106) The analyzing unit 113 calculates the heart rate based on the likeliest model. As one example, the analyzing unit 113 calculates the average time (d₁, d₂) spent in specified states (S₁ and S₂) corresponding to waveforms with little body movement noise from a state series {s*[t]} (see Expression (10) below) estimated by a Viterbi algorithm for the model information (A,Q,r) of the likeliest model. The analyzing unit 113 then calculates the heart rate HR according to Expression (11) below. Here, d₁ is the average time spent in state S₁, that is, the average value of the time between entering state S₁ and exiting to another state. Similarly, d₂ is the average time spent in state S₂, that is, the average value of the time between entering state S₂ and exiting to another state.

$$\begin{matrix}\{ s * [ t ] \} = {arg max }_{s} [ P ( \{ x [ t ] \} , \{ s [ t ] \} ; A , Q , r ) ] & ( 10 ) \\ HR = \frac{60}{{d}_{1} + {d}_{2}} & ( 11 )\end{matrix}$$

As another example method of calculating the heart rate, it is possible to use a method where a heart rate is tied to model information itself. Model information corresponding to a plurality of heart rates that are set in advance is provided (for example, model information for a heart rate of 30 bpm, model information for a heart rate of 31 bpm, . . . , model information for a heart rate of 180 bpm) and the analyzing unit 113 specifies the heart rate that is tied to the selected model information.

The heart rate calculated by the analyzing unit 113 is transmitted by the transmitting unit 114 to the information processing apparatus 201 or the like. When the processing in S106 is completed, the series of processes depicted in FIG. 8 ends.

Label Series Generation Process

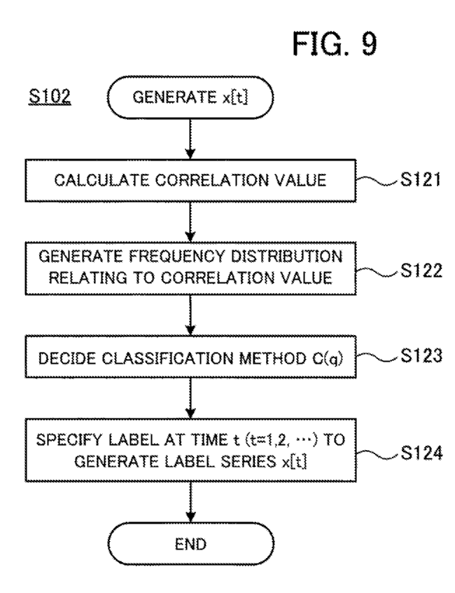

Here, processing relating to the generation of the label series x[t], that is, processing corresponding to S102, will be described further with reference to FIG. 9. FIG. 9 is a flowchart depicting an operation (or “label series generation process”) of the heart rate estimating apparatus according to the second embodiment.

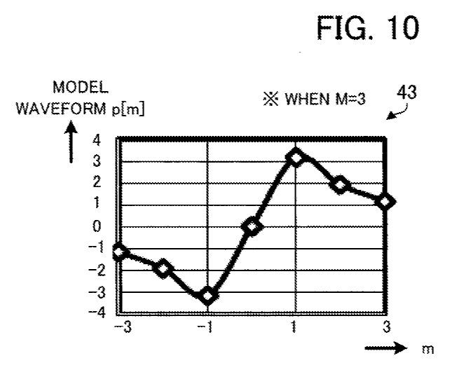

(S121) The analyzing unit 113 calculates a correlation value q[t] from the pulse wave data n[t]. The correlation value q[t] is given by Expression (12) below. Here, p[m] is a model waveform (see FIG. 10) in which many waveforms that are included in an ideal pulse wave that does not include body movement noise are modeled. FIG. 10 is a graph depicting one example of a model waveform used to calculate a correlation value. The graph 43 depicted in FIG. 10 depicts a model waveform. The width (or “correlation width”) of the model waveform is set at around 30 points, for example. When the sampling interval of the pulse wave data n[t] acquired from the pulse wave sensor 101 is 30 Hz, the width of the model waveform corresponds to around one second, for example.

$$q [ t ] = {\sum }_{m = - M}^{M} p [ m ] \cdot n [ t - m ] \qquad ( 12 )$$

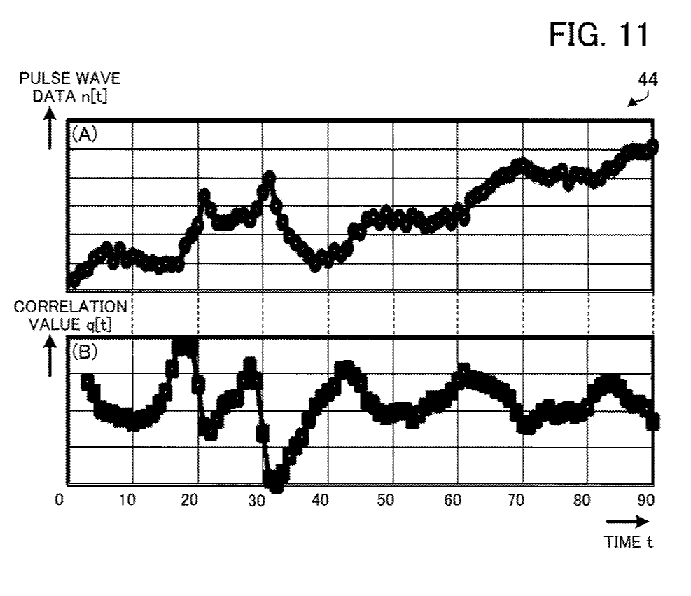

Note that the coefficients of a high frequency filter such as a differential filter may be used as p[m]. Calculation of the correlation between the pulse wave data n[t] and the model waveform corresponds to differentiation of the waveform indicated by the pulse wave data n[t], for example. When pulse wave data n[t] like that depicted in (A) of FIG. 11 is provided, the correlation value q[t] calculated according to Expression (12) given above is as depicted in (B) of FIG. 11. FIG. 11 depicts the relationship between the pulse wave data and the correlation value. The graphs 44 in FIG. 11 depict the correlation value q[t] corresponding to the pulse wave data n[t].

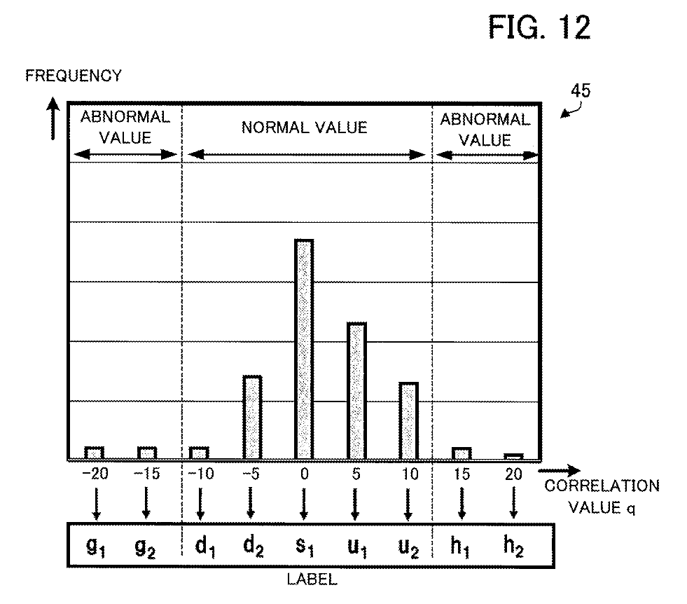

(S122) The analyzing unit 113 generates a frequency distribution (see FIG. 12) relating to the correlation value q[t] calculated in S121. FIG. 12 depicts one example of a classification method based on a frequency distribution relating to the correlation value. The graph 45 depicted in FIG. 12 expresses appearance frequencies in the correlation value q[t]. For the frequency distribution depicted in FIG. 12, the horizontal axis is the correlation value q and the vertical axis is the frequency. From the definition (see Expression (12) described above) of the correlation value q, the closer the correlation value q is to zero, the higher the probability of a normal value, which is close to the model waveform and has little body movement noise. On the other hand, the further the correlation value q from zero, the higher the probability of an abnormal value, which is far from the model waveform and has been greatly affected by body movement noise.

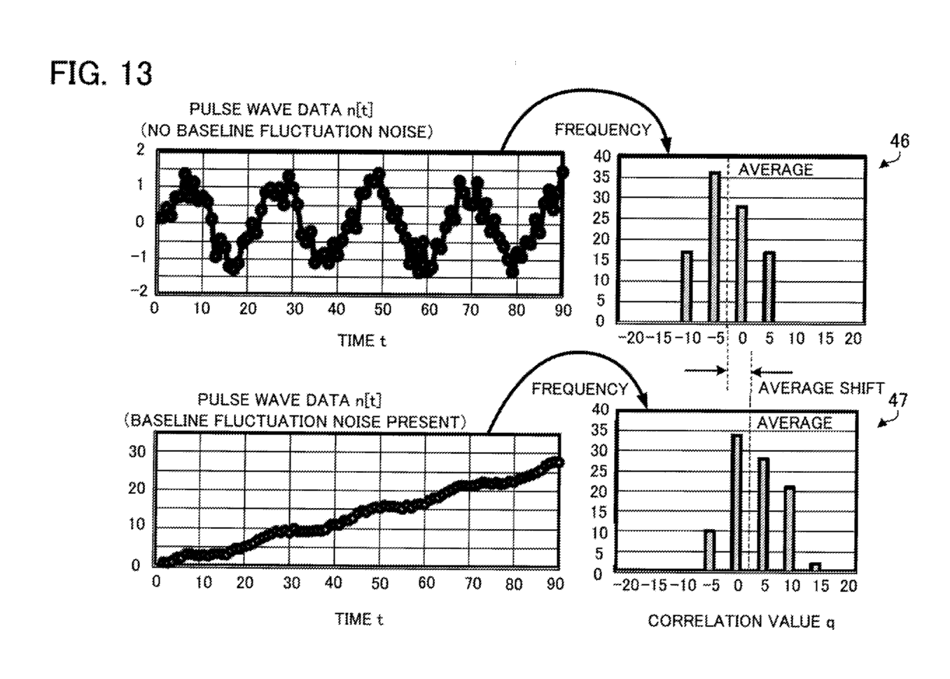

Note that the effect of baseline fluctuation noise is expressed by the average of the frequency distribution. As one example, when baseline noise is incorporated in the pulse wave data n[t], as depicted in FIG. 13, the average of the frequency distribution shifts by an amount equivalent to the inclination component of the baseline fluctuation noise. FIG. 13 depicts how a correlation value is affected by the presence or absence of baseline fluctuation noise. When a graph 46 and the graph 47 in FIG. 13 are compared, the graph 47 has a larger average correlation value due to the rightward-rising baseline fluctuation noise.

(S123) The analyzing unit 113 decides a classification method C(q) for the correlation value q from the frequency distribution generated in S122. That is, the analyzing unit 113 defines the boundary for dividing between normal values and abnormal values described earlier based on the frequency distribution. At this time, the analyzing unit 113 further divides the range of normal values and the range of abnormal values into smaller ranges and assigns a label to each range.

The analyzing unit 113 calculates an average μ of the correlation value q[t] in accordance with Expression (13) below. The analyzing unit 113 calculates the variance σ² of the correlation value q[t] in accordance with Expression (14) below. The analyzing unit 113 then decides ranges of the correlation value q to be assigned the labels g₁, g₂, d₁, d₂, s₁, u₁, u₂, h₁, and h₂ based on Expression (15) below.

$$\begin{matrix}µ = E [ \{ q [ t ] \} ] = \frac{1}{N} {\sum }_{t = 1}^{N} q [ t ] & ( 13 ) \\ {σ}^{2} = V [ \{ q [ t ] \} ] = \frac{1}{N} {\sum }_{t = 1}^{N} {( q [ t ] - µ )}^{2} & ( 14 ) \\ C ( q ) = \{ \begin{matrix}{g}_{1} ( q - µ \leq - 2 σ ) \\ {g}_{2} ( - 2 σ < q - µ \leq - \frac{3}{2} σ ) \\ {d}_{1} ( - \frac{3}{2} σ < q - µ \leq - σ ) \\ {d}_{2} ( - σ < q - µ \leq - \frac{1}{2} σ ) \\ {s}_{1} ( - \frac{1}{2} σ < q - µ \leq \frac{1}{2} σ ) \\ {u}_{1} ( \frac{1}{2} σ < q - µ \leq σ ) \\ {u}_{2} ( σ < q - µ \leq \frac{3}{2} σ ) \\ {h}_{1} ( \frac{3}{2} σ < q - µ \leq 2 σ ) \\ {h}_{2} ( 2 σ < q - µ )\end{matrix} & ( 15 )\end{matrix}$$

From the definition in Expression (15), g₁, g₂, h₁, and h₂ are labels corresponding to when the correlation value q greatly diverges from the center and indicate abnormal values (values likely to appear when body movement noise is present). On the other hand, d₁, d₂, s₁, u₁, and u₂ are labels corresponding to when the correlation value q does not greatly diverge from the center, and indicate normal values. The relationship between ranges of the correlation value q and the labels assigned to these ranges (the relationship in Expression (15) described above) is referred to as “the classification method C(q)”.

As described earlier, when body movement noise including a large amount of low frequency components is incorporated in the pulse wave data, the average of the correlation value q shifts. If the classification described above were performed without consideration to this shift, there would be the risk of classification of the correlation value q not being performed appropriately in keeping with the features of the waveform. However, like the Expression (15) described above, by deciding the classification method C(q) using the standard deviation with a value produced by subtracting the average p from the correlation value q as a standard, it is possible to appropriately consider the shift and thereby suppress the risk mentioned above.

(S124) The analyzing unit 113 compares the correlation value q[t] and the classification method C(q), specifies the label at the time t (t=1, 2, . . . ), and generates the label series x[t] (see Expression (16) below). When the processing in S124 is completed, the series of processes depicted in FIG. 9 ends.

x[t]=C(q[t]) (16)

Calculation of Likelihood

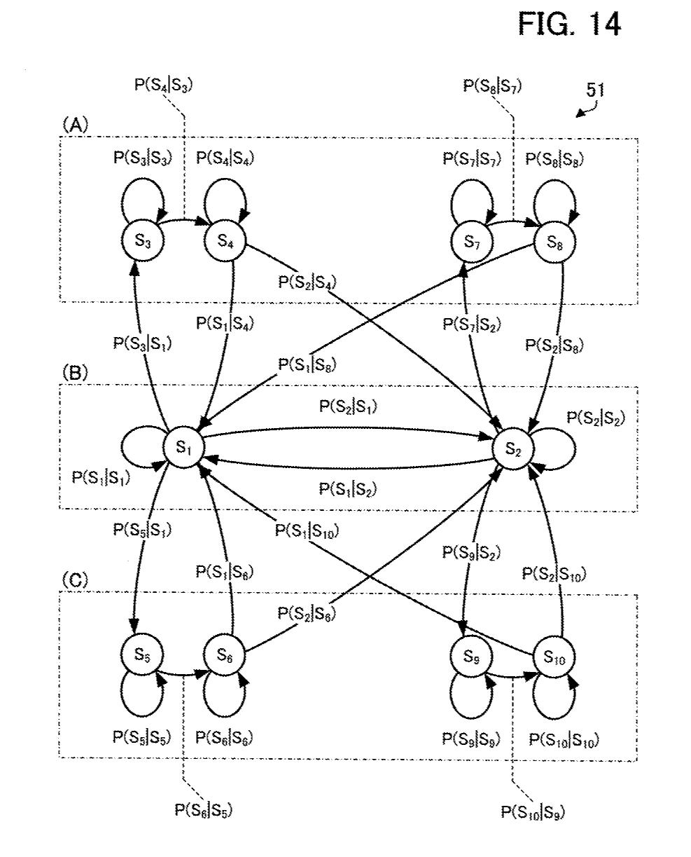

An example of a hidden Markov model and calculation of likelihood by the analyzing unit 113 will now be described further with reference to FIG. 14. FIG.

is a state transition chart that defines a hidden Markov model used to estimate heart rate.

In the state transition chart 51 illustrated in FIG. 14, ten states S₁, S₂, . . . , S₁₀ (that is, N_{c}=10) are given, and the state transition probabilities P(Sⱼ|Sᵢ)(where i,j=1,2, . . . , 10) are expressed by the arrows. Note that the state transition chart 51 in FIG. 14 is one example, and the combinations of arrows that define combinations of states where the transition probability is not zero can be transformed. Although the state transition chart 51 in FIG. 14 includes twenty-eight state transitions as typical state transitions, it is also possible to further include other state transitions (for example, a transition from state S₄ to state S₇).

The states in (A) of FIG. 14 are states where abnormal values are likely to be observed (states where upwardly spiking noise is likely). The states in (B) of FIG. 14 are states where normal values are likely to be observed. The states in (C) of FIG. 14 are states where abnormal values are likely to be observed (states where downwardly spiking noise is likely). That is, S₁ and S₂ express states where label v∈{d₁, d₂, s₁, u₁, u₂} is likely to be observed, and S₃, S₄, S₅, S₆, S₇, S₈, S₉, and S₁₀ express states where label v∈{g₁, g₂, h₁, h₂} is likely to be observed.

The transition probability between the states Sᵢ and Sⱼ is given by a matrix A (model information) expressed by Expression (17) below. The probability of the label vⱼ being observed in a state Sᵢ is given by a matrix Q (model information) expressed by Expression (18) below. The label v₁ is expressed by Expression (19) below. The probability that the initial state (t=1) is Sⱼ is given by a vector r (model information) expressed by Expression (20) below. This model information is stored in the storage unit 111.

$$\begin{matrix}A = ( \begin{matrix}P ( {S}_{1} \vert {S}_{1} ) & … & P ( {S}_{10} \vert {S}_{1} ) \\ ⋮ & ⋱ & ⋮ \\ P ( {S}_{1} \vert {S}_{10} ) & … & P ( {S}_{10} \vert {S}_{10} )\end{matrix} ) & ( 17 ) \\ Q = ( \begin{matrix}P ( {v}_{1} \vert {S}_{1} ) & … & P ( {v}_{9} \vert {S}_{1} ) \\ ⋮ & ⋱ & ⋮ \\ P ( {v}_{1} \vert {S}_{10} ) & … & P ( {v}_{9} \vert {S}_{10} )\end{matrix} ) & ( 18 ) \\ ( {v}_{1} , {v}_{2} , … , {v}_{9} ) = ( {g}_{1} , {g}_{2} , … , {h}_{1} , {h}_{2} ) & ( 19 ) \\ r = ( P ( {S}_{1} ) , P ( {S}_{2} ) , … , P ( {S}_{10} ) ) & ( 20 )\end{matrix}$$

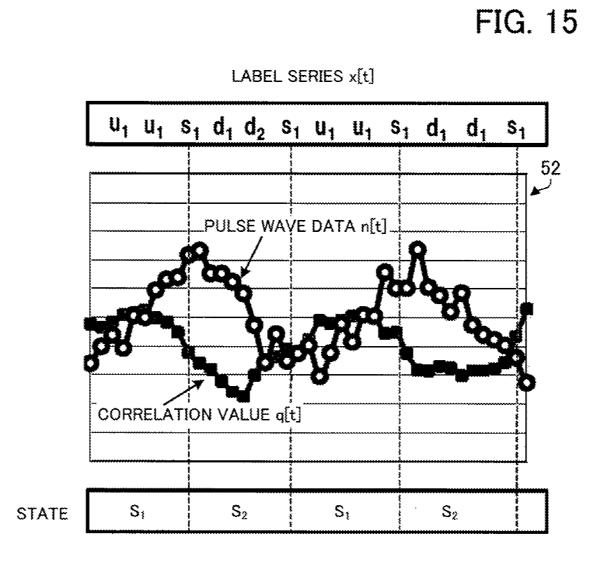

The relationship between the pulse wave data n[t], the correlation value q[t], the label series x[t], and the states is as depicted in FIGS. 15 to 17. FIG. 15 depicts the relationship between the pulse wave data, the correlation value, the label series, and the states (when there is little body movement noise). The graph 52 depicted in FIG. 15 expresses pulse wave data n[t] with little body movement noise and the correlation value q[t] corresponding to this pulse wave data n[t]. The label series x[t] calculated from the correlation value q[t] does not include a label corresponding to an abnormal value and expresses periodic variation in a range of normal values. A hidden Markov model assumes that state transitions are unknown (or “hidden”) and estimates state transitions with the highest probability that the label series x[t] will be outputted. Therefore, state transitions between the states S₁ and S₂ where normal values are likely to be observed alternately appear in the string of state transitions estimated from the label series x[t].

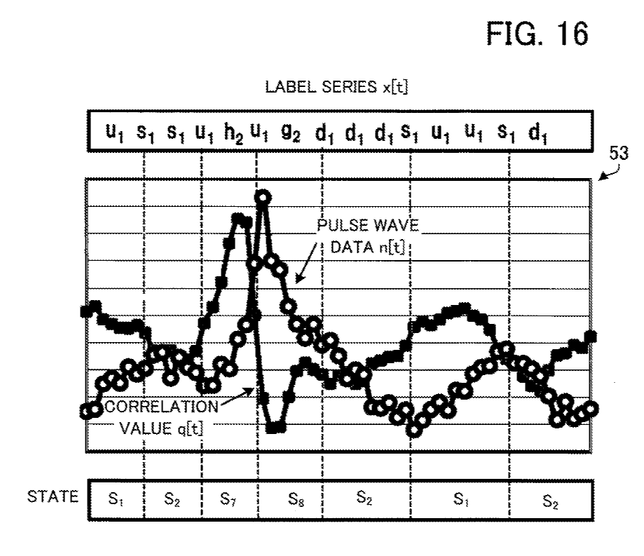

FIG. 16 depicts the relationship between the pulse wave data, the correlation value, the label series, and the states (when there is upwardly spiking body movement noise). The graph 53 depicted in FIG. 16 expresses pulse wave data n[t] including upwardly spiking body movement noise and the correlation value q[t] corresponding to this pulse wave data n[t]. The label series x[t] calculated from the correlation value q[t] includes labels corresponding to upwardly spiking abnormal values that are mixed into periodic variations within a range of normal values. Therefore, state transitions to the states S₇ and S₈ where upwardly spiking abnormal values are likely to be observed appear in a string of state transitions estimated from the label series x[t].

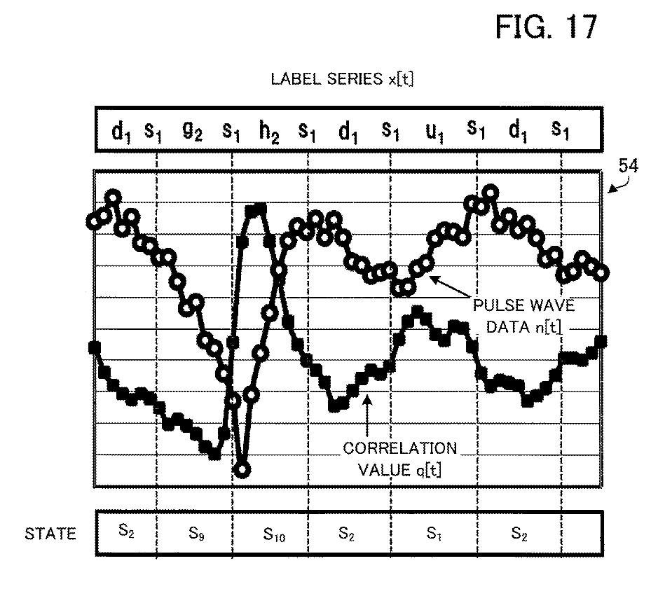

FIG. 17 depicts the relationship between the pulse wave data, the correlation value, the label series, and the states (when there is downwardly spiking body movement noise). The graph 54 depicted in FIG. 17 expresses pulse wave data n[t] including downwardly spiking body movement noise and the correlation value q[t] corresponding to this pulse wave data n[t]. The label series x[t] calculated from the correlation value q[t] includes labels corresponding to downwardly spiking abnormal values that are mixed into periodic variations within a range of normal values. Therefore, state transitions to the states S₉ and S₁₀ where downwardly spiking abnormal values are likely to be observed appear in a string of state transitions estimated from the label series x[t]. In this way, by classifying peaks in the pulse wave and body movement noise using labels and assigning a state to each, the risk of body movement noise being erroneously recognized as peaks in a pulse wave is suppressed.

Generation Process for Model Information

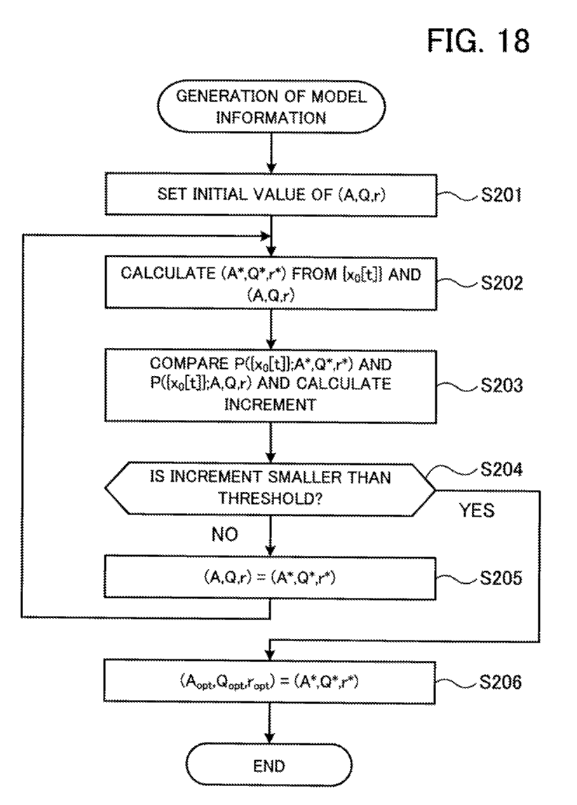

Here, the generation process for the model information (A,Q,r) will be described with reference to FIG. 18. FIG. 18 is a flowchart depicting the operation (or “generation process for model information”) of the heart rate estimating apparatus according to the second embodiment.

The model information uses the pulse wave data n₀(t) that includes abnormal value components and is prepared in advance as learning samples, and is calculated using an expectation maximization method (for example, a Baum-Welch algorithm) based on a label series x₀(t) generated from the pulse wave data n₀(t). That is, the generating process for the model information corresponds to a process that calculates (Aₒₚₜ,Qₒₚₜ,rₒₚₜ) given by Expression (21) below as the model information. Note that a Baum-Welch algorithm is an algorithm used to estimate unknown parameters of a hidden Markov model.

$$( {A}_{opt} , {Q}_{opt} , {r}_{opt} ) = {arg max}_{( A , Q , r )} \{ P ( \{ {x}_{0} [ t ] \} ; A , Q , r ) \} \qquad ( 21 )$$

Note that although the model information may be calculated in advance by a computer aside from the heart rate estimating apparatus 100 (for example, a server computer or a cloud system) and stored in the storage unit 111 of the heart rate estimating apparatus 100, the description here assumes that the analyzing unit 113 generates the model information.

(S201) The analyzing unit 113 sets the initial values of the model information (A,Q,r) of the pulse wave model Ω. As examples, the initial values of the model information (A,Q,r) may be values decided in advance or may be values decided at random. The analyzing unit 113 generates the label series x₀(t) from the pulse wave data n₀(t) prepared as learning samples using the same method as the method of generating the label series x[t] depicted in FIG. 9.

(S202) The analyzing unit 113 uses the label series x₀(t) generated in S201 and the model information (A,Q,r) to calculate candidates (A*,Q*,r*) for the model information. The element in the i^{th} row and j^{th} column of the matrix A* is given by Expression (22) below. The element in the j^{th} row and k^{th} column of the matrix Q* is given by Expression (23) below. The i^{th} element in the vector r* is given by Expression (24) below. The element aᵢⱼ included in Expression (22) below is the i^{th} row and j^{th} column of the matrix A set in S201.

$$\begin{matrix}{a}_{ij}^{*} = \frac{{\sum }_{t = 1}^{n - 1} {α}_{t} ( i ) \cdot {a}_{ij} \cdot P ( {x}_{0} [ t + 1 ] \vert {S}_{j} ) \cdot {β}_{t + 1} ( j )}{{\sum }_{t = 1}^{n - 1} {α}_{t} ( i ) \cdot {β}_{t} ( i )} & ( 22 ) \\ {q}_{jk}^{*} = \frac{{\sum }_{t = 1}^{n - 1} {α}_{t} ( j ) \cdot δ ( {x}_{0} [ t ] , {S}_{k} ) \cdot {β}_{t} ( j )}{{\sum }_{t = 1}^{n - 1} {α}_{t} ( j ) \cdot {β}_{t} ( j )} & ( 23 ) \\ {r}_{i}^{*} = \frac{{α}_{1} ( i ) \cdot {β}_{1} ( i )}{{\sum }_{j = 1}^{Nc} {α}_{n} ( j )} & ( 24 )\end{matrix}$$

Also, αₜ(t=1,2, . . . , n) is obtained by a forward algorithm that sets an initial value α₁ in Expression (25) below and recursively calculates αₜ in Expression (26) below. β_{L} (t=1,2, . . . , n) is obtained by a backward algorithm that sets an initial value βₙ in Expression (27) below and recursively calculates βₜ in Expression (28) below. δ(·) included in Expression (23) below is the Kronecker Delta.

$$\begin{matrix}{α}_{1} ( i ) = {r}_{i} \cdot P ( {x}_{0} [ 1 ] \vert {S}_{i} ) & ( 25 ) \\ {α}_{t} ( j ) = [ {\sum }_{i = 1}^{Nc} {α}_{t - 1} ( i ) \cdot {a}_{ij} ] \cdot P ( {x}_{0} [ t ] \vert {S}_{j} ) & ( 26 )\end{matrix}$$

(where i=1,2, . . . , N_{c} and t=2,3, . . . , n)

$$\begin{matrix}{β}_{n} ( i ) = 1 & ( 27 ) \\ {β}_{t} ( i ) = {\sum }_{j = 1}^{Nc} {a}_{ij} \cdot P ( {x}_{0} [ t + 1 ] \vert {S}_{j} ) \cdot {β}_{t + 1} ( j ) & ( 28 )\end{matrix}$$

(where i=1,2, . . . , N_{c} and t=2,3, . . . , n−1)

(S203) The analyzing unit 113 calculates P({x₀[t]};A*,Q*,r*) and P({x₀[t]};A,Q,r) based on Expression (29) below. The analyzing unit 113 then compares P({x₀[t]};A*,Q*,r*) and P({x₀[t]};A,Q,r) and calculates the increment (P({x₀[t]};A*, Q*, r*)−P({x₀[t]};A, Q, r)).

P({x₀[t]}; A, Q, r)=Σᵢ₌₁^{N}^{_{c}}αₙ(i) (29)

(S204) The analyzing unit 113 compares the increment calculated in S203 and a threshold set in advance and determines whether the increment is smaller than the threshold. When the increment is smaller than the threshold, the processing proceeds to S206. On the other hand, when the increment is not smaller than the threshold, the processing proceeds to S205.

(S205) The analyzing unit 113 sets the candidate (A*,Q*,r*) of the model information calculated in S202 as the new model information (A,Q,r). When the processing in S205 is completed, the processing proceeds to S202.

(S206) The analyzing unit 113 sets the candidate (A*,Q*,r*) of the model information calculated in S202 as the model information (Aₒₚₜ,Qₒₚₜ,rₒₚₜ) and stores the information in the storage unit 111. When the processing in S206 is completed, the series of processes depicted in FIG. 18 ends. In this way, in the processing in FIG. 18, since pulse wave data n₀(t) that includes body movement noise is used as learning samples, model information that considers body movement noise is obtained.

2-4 Modifications

Although a framework where the heart rate estimating apparatus 100 estimates the heart rate from pulse wave data has been described so far, it is also possible to transmit pulse wave data from a wearable device attached to the body to an information processing apparatus and to estimate the heart rate at the information processing apparatus.

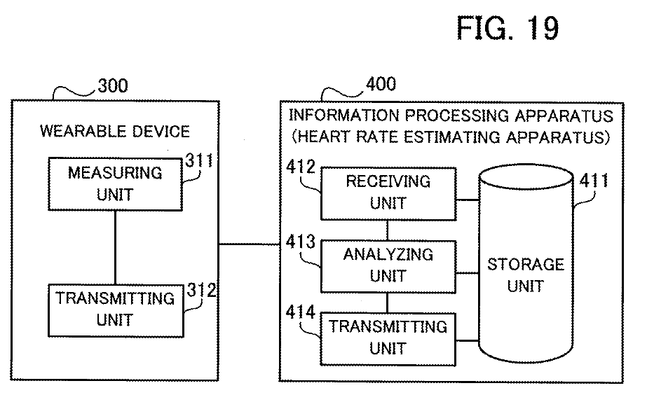

FIG. 19 depicts another example of a system according to the second embodiment. This other example system includes a wearable device 300 and an information processing apparatus 400. The wearable device 300 is a band-type device that wraps around the wrist, for example. The information processing apparatus 400 is a smartphone, a PC, a server computer, or the like.

The wearable device 300 can be realized using the same hardware as the heart rate estimating apparatus 100 in FIG. 3. The information processing apparatus 400 can be realized using the same hardware as the information processing apparatus 201 in FIG. 4. The wearable device 300 and the information processing apparatus 400 may be located near one another. One conceivable example is where the information processing apparatus 400 is a smartphone or PC belonging to the same person as the wearable device 300. In this example, both devices may communicate using a near-field wireless communication technology. To do so, the RF circuit 105 and the communication interface 217 may be equipped with a near-field wireless communication technology.

The wearable device 300 and the information processing apparatus 400 may alternatively be remotely located. One conceivable example is where the information processing apparatus 400 is a server computer in a cloud system. Here, both devices may communicate via a base station using a wide-area wireless communication technology. To do so, the RF circuit 105 may be equipped with a near-field wireless communication technology and the communication interface 217 may be equipped with a wired communication technology.

The wearable device 300 includes a measuring unit 311 and a transmitting unit 312. The measuring unit 311 corresponds to the measuring unit 112 in FIG. 7. The measuring unit 311 generates pulse wave data using the pulse wave sensor 101. The transmitting unit 312 transmits pulse wave data to the information processing apparatus 400.

The information processing apparatus 400 includes a storage unit 411, a receiving unit 412, an analyzing unit 413, and a transmitting unit 414. The information processing apparatus 400 can also be referred to as a “heart rate estimating apparatus”. The storage unit 411 corresponds to the storage unit 111 in FIG. 7. The storage unit 411 stores the model information of the pulse wave model Ω. The receiving unit 412 receives the pulse wave data from the wearable device 300. The analyzing unit 413 corresponds to the analyzing unit 113 in FIG. 7. The analyzing unit 413 analyzes the pulse wave data received by the receiving unit 412 and estimates the heart rate. The transmitting unit 414 transmits the heart rate estimated by the analyzing unit 413 to the wearable device 300 or another information processing apparatus (for example, a smartphone or a PC). However, the information processing apparatus 400 may output the estimated heart rate to an output device (as examples, a display or a printer) of the information processing apparatus 400. The information processing apparatus 400 may also store the estimated heart rate in the storage unit 411.

This completes the description of the second embodiment.

As described above, by using the heart rate estimating apparatus 100 according to the second embodiment, it is possible to precisely estimate the heart rate, even when body movement noise is incorporated in the pulse wave data. Also, since heart rate is estimated based on a hidden Markov model, compared to a heart rate estimating apparatus that estimates a heart rate using a DFT, it is possible to reduce the sensor operating time and thereby possible to suppress power consumption.

According to the above embodiments, it is possible to improve the precision of estimating heart rate from pulse wave data that includes noise.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A heart rate estimating apparatus comprising:
a sensor that generates pulse wave data indicating a pulse wave of a body;
a memory that stores a plurality of state transition models, the plurality of state transition models each representing a plurality of states including a normal state and an abnormal state corresponding to noise, transition probabilities between the plurality of states, and output probabilities of each of a plurality of different symbols being observed in each of the plurality of states, wherein at least one of the transition probabilities and the output probabilities differs between the respective state transition models; and
a processor that generates a symbol string that indicates a time series of changes in the pulse wave from the pulse wave data using the plurality of different symbols, calculates a fit between the symbol string and each of the plurality of state transition models, and calculates a heart rate estimated from the pulse wave data based on a state transition model that has been selected out of the plurality of state transition models in accordance with the fit.

2. The heart rate estimating apparatus according to claim 1,
wherein the plurality of state transition models are state transition models generated using a plurality of sample sets of pulse wave data including different noise.

3. The heart rate estimating apparatus according to claim 1,
wherein each of the plurality of the state transition models includes at least two normal states, which are cyclically transitioned between when noise is not present, and at least two abnormal states, which are branches from the at least two normal states.

4. The heart rate estimating apparatus according to claim 1,
wherein the processor classifies patterns of changes in the pulse wave per unit time into a plurality of classes in keeping with appearance frequency in the pulse wave data and assigns a different symbol to each of the plurality of classes.

5. The heart rate estimating apparatus according to claim 1,
wherein a model heart rate is associated with each of the plurality of state transition models, and
the processor specifies the heart rate based on the model heart rate associated with the selected state transition model.

6. A heart rate estimating system comprising:
a terminal apparatus including a sensor that generates pulse wave data indicating a pulse wave of a body; and
an estimating apparatus including a processor that receives the pulse wave data from the terminal apparatus, generates a symbol string that indicates a time series of changes in the pulse wave from the pulse wave data using a plurality of different symbols, calculates a fit between the symbol string and each of a plurality of state transition models stored in a memory, and calculates a heart rate estimated from the pulse wave data based on a state transition model that has been selected out of the plurality of state transition models in accordance with the fit,
wherein the plurality of state transition models each represent a plurality of states including a normal state and an abnormal state corresponding to noise, transition probabilities between the plurality of states, and output probabilities of each of the plurality of different symbols being observed in each of the plurality of states, and at least one of the transition probabilities and the output probabilities differs between the respective state transition models.

7. A non-transitory computer-readable storage medium storing a heart rate estimating program, the heart rate estimating program that causes a computer including a sensor, a memory, and a processor to perform a procedure comprising:
acquiring, by the processor, pulse wave data indicating a pulse wave of a body from the sensor;
generating, by the processor, a symbol string that indicates a time series of changes in the pulse wave from the pulse wave data using a plurality of different symbols; and
calculating, by the processor, a fit between the symbol string and each of a plurality of state transition models stored in the memory, and calculating a heart rate estimated from the pulse wave data based on a state transition model that has been selected out of the plurality of state transition models in accordance with the fit,
wherein the plurality of state transition models each represent a plurality of states including a normal state and an abnormal state corresponding to noise, transition probabilities between the plurality of states, and output probabilities of each of the plurality of different symbols being observed in each of the plurality of states, and at least one of the transition probabilities and the output probabilities differs between the respective state transition models.
